(19) United States
(12) Patent Application Publication
(10) Pub. No.: US 2023/0022136 A1
(43) Pub. Date: Jan. 26, 2023

(54) DIFFUSIVITY CONTRAST AGENTS FOR MEDICAL IMAGING

(71) Applicant: Jinghua WANG, Mason, OH (US)

(72) Inventor: Jinghua Wang, Mason, OH (US)

(21) Appl. No.: 17/908,086

(22) PCT Filed: Jul. 13, 2021
(86) PCT No.: PCT/US2021/070867

§ 371 Date: Aug. 30, 2022

Related U.S. Application Data
(60) Provisional application No. 63/068,812, filed on Aug. 21, 2020.

(51) Int. Cl. A61B 6/00; A61K 49/06

(52) U.S. Cl. CPC A61B 6/488; A61K 49/06; A61B 8/481

(57) ABSTRACT

A method for medical imaging using a diffusivity contrast agent is provided. The method includes obtaining the diffusivity contrast agent having specific diffusivity, wherein the diffusivity contrast agent is configured to pass a biological barrier of a subject; administering to the subject a detectable dose of the diffusivity contrast agent for at least one medical imaging modality, where the medical imaging modality includes one or more of MRI, PET, SPECT, CT, x-ray, optical imaging and ultrasound; acquiring one or more post-contrast images for a region of interest after the administration of the diffusivity contrast agent, wherein said post-contrast images include changes in diffusivity of the region of interest compared to one or more images acquired without said diffusivity contrast agent; and characterizing a transport of the diffusivity contrast agent in the region of interest based on the changes in diffusivity of the region of interest.

### CROSS-REFERENCE TO RELATED APPLICATIONS

This application claims priority to the benefit of the filing date of U.S. Provisional Application No. 63/068,812, entitled “New contrast agent for medical imaging” filed on Aug. 21, 2020, which is hereby incorporated by reference in its entirety.

### BACKGROUND

### 1. Field of the Invention

The present invention relates to diffusivity contrast agents that are characterized by diffusion-enhanced contrast in medical imaging, including by x-rays, ultrasound tomography, computed tomography (CT), positron emission tomography (PET), single photo-emitting computed tomography (SPECT), optical imaging, and magnetic resonance imaging (MRI).

### 2. Description of the Related Art

Biological barriers are defined as living organisms which serve to protect the body from invading pathogens and xenogens. The barriers include cell membrane, nuclear membrane, blood-tissue barrier (BTB), skin, mucosal membranes, etc. BTB includes at least one of blood-brain barrier (BBB), but not limited to, blood-cerebrospinal fluid (CSF) barrier, the blood-retinal barrier, the blood-testis barrier, and blood-brain tumor barrier (BBTB). One of the most important BTB functions is to separate vital organs from external cues and harmful substances in the environment. The barrier safeguards key physiological processes. For example, BBB provides nutrient substances (e.g. glucose, amino acids, lactate, pyruvate, vitamins, metals, peptides and proteins) and prevents harmful substances (e.g. bacteria, viruses, and potentially harmful large or hydrophilic molecules) from entering the brain of healthy people. BBB dysfunction has been shown to play a direct key role in many brain diseases and neurological disorders, such as brain tumors, traumatic brain injury, stroke, chronic vascular disease, Parkinson's disease, vascular cognitive impairment, multiple sclerosis, Alzheimer's disease and dementia. BBB disruption is also a common pathological finding in many psychiatric disorders including schizophrenia, autism spectrum disorder and mood disorders. Moreover, an intact BBB may restrict the delivery of certain therapeutic substances to the brain. For example, BBB may prevent or delay the transport of chemotherapy drugs (except for some small molecule drugs with molecular weights of less than 400 Dalton) or hydrophobic molecules. As a consequence, the normal action of BBB can block drugs entering the brain and introduces difficulties for drug delivery, therefore monitoring drug delivery is deemed highly challenging, if not unfeasible.

Today, contrast-based studies are commonplace in clinical and research-based applications. In 2015, Beckett et al. reported “half of the approximately 76 million computed tomographic (CT) and 34 million magnetic resonance imaging (MRI) examinations performed each year include the use of intravenous contrast agents.” Katrina R. Beckett et al., Safe Use of Contrast Media: What the Radiologist Needs to Know, 35 RadioGraphics 1738-1750 (October 2015).

Contrast agents are used to enhance image contrasts and allow radiologists to distinguish normal conditions from abnormal ones easily. There are millions of radiological examinations with contrast agents worldwide annually. Various image modalities, such as x-rays, ultrasound tomography, computed tomography (CT), positron emission tomography (PET), single photo-emitting computed tomography (SPECT), and magnetic resonance imaging (MRI), are available to qualitatively and/or quantitatively measuring the transport of contrast agents across BTB in vivo using contrast agents. The transport of the contrast agents across BTB is zero or very small under normal physiological conditions, and become large for pathological tissues. The contrast agents used for estimating BTB are either exogenous or endogenous. Over past decades, only a small number of contrast agents have been approved by FDA for clinical practices. There remains a lot of challenges for the development and application of contrast agents. For example, the use of the CT and x-rays contrast agents (e.g., iodinated contrast agent) are limited by some disadvantages, such as ionizing radiation, poor soft tissue contrast and an increasing risk for adverse reactions. In contrast, MRI contrast agents provide better soft tissue contrast without ionizing radiation. Gadolinium-based MRI contrast agents are used in about 40% of all MRI exams and in about 60% of neuro MRI exams. However, recent studies suggest that intravenous administration of Gd-based MR contrast agents is associated with dose-dependent deposition in neuronal tissues that is unrelated to renal function, age, or interval between exposure and death. It is still unknown whether these gadolinium deposits are harmful or can lead to adverse health effects. Therefore, the Food and Drug Administration suggested that doctors limit the use of contrast agents unless additional information provided by the contrast agents is necessary. Chemical exchange saturation transfer (CEST) agents that are a newest class of MR contrast agents is based on protons exchanging between one type of molecule and another. For example, glucose is used as an exogenous CEST agent to overcome the potential risk of conventional MRI contrast agents. Various contrast agents used for medical imaging over the past decade, are disclosed in the following references:

WO2002006287A2 and U.S. Pat. No. 6,656,450B2 to Timothy J. Hubin and Thomas J. Meade disclosed a novel MRI contrast agent for T₁ or T₂*-weighted MR imaging.

WO2006114738A2 and U.S. Pat. No. 8,734,761 to Nicolaas P. Willard et al. disclosed MRI contrast agents comprising CEST active paramagnetic complex for determining local pH, temperature, oxygen concentration or other metabolites in a patient's body.

WO2008022349A2 and U.S. Pat. No. 8,497,246B2 to William M. Pardridge and Ruben J. Boado disclosed methods for delivery of systemic protein pharmaceuticals (such as insulin receptor, transferrin receptor, leptm receptor, lipoprotein receptor, or the IGF receptor) across the blood-brain barrier into the central nervous system (CNS).

WO2008130439A1 and U.S. Pat. No. 8,758,723B2 to David J. Yang et al. disclosed N4 compounds as contrast agents for PET and SPECT.

WO2009000777A2 and U.S. Pat. No. 9,682,159B2 to Ulrike Wiebelitz disclosed the use of a combination of several contrast agents having different properties with respect to imaging representation.

U.S. Pat. No. 9,017,646B2 to Jun-Ming Shih et al. disclosed a fluorescent substance as a contrast agent in the fluorescent microscopy imaging for evaluating the BBB permeability.

WO2014205338A2 and U.S. Patent Application Publication No. 20160120893A1 to Chenghua Gu and Ayal Ben-Zvi disclosed an agonist of a gene or gene expression product to modulating the BBB permeability for therapeutic purposes.

WO2014186737A1 and U.S. patent Ser. No. 10/188,754B2 to Xing Yang et al. disclosed beta-hydroxycarboxylate and beta-aminocarboxylate derivatives as a contrast agent for CEST based MRI or frequency labeled exchange imaging.

WO2015195501A1 and U.S. Patent Application Publication No. 20170095578 A1 to Adib Raphael Karam and Andrew Karellas disclose a gadoxeate disodium as a contrast agent for making images such as CT scans. The contrast agent exhibits the paramagnetic property and strong x-ray absorption. Because of their x-ray absorption properties, gadolinium-based agents have been proposed as an alternative to iodinated contrast agents for x-ray planar angiographic and CT.

WO2017049411A1 and U.S. patent Ser. No. 10/398,753B2 to Philippe Patrick Monnier et al. disclosed compositions (RGMa, soluble RGMa, and functional fragments and variants thereof, RGMc, soluble RGMc, and functional fragments and variants thereof, and Neogenin peptides including 4 Ig.) for modulating the blood-brain barrier permeability for the treatment of diseases and conditions and to facilitate the delivery of agents to the brain, as well as methods and compositions for promoting re-myelination and preventing de-myelination.

WO2017098044A1 and U.S. Patent Application Publication No. 2021/0024500 to Valeria Boi, et al. disclosed new class of dimeric macrocycles capable of chelating paramagnetic metal ions and the use thereof as contrast agents.

WO2018053460 to Gabriel E. Sanojya et al. disclosed a paramagnetic polymer composition as a contrast agent in MRI.

WO2019012530A1 and U.S. Patent Application Publication No. 20200405885 to Shai Berlin disclosed a hybrid molecule comprising at least one contrast agent, and at least one substrate of a self-labeling enzyme for clinical anatomical imaging.

U.S. Patent Application Publication No. 20200062791 to Mikkel Jacob Thaning et al. disclosed manganese (II) complexes as an MRI contrast agent.

WO2020127154 to Silvio Aime et al. disclosed pharmaceutical compositions comprising a gadolinium complex and a water-soluble polyarylene additive useful as contrast agents in MRI.

WO2020243585A1 and U.S. Patent Application Publication No. 20200376143 to Michael Scott Echols disclosed a method and system for preserving a subject and then introducing perfusible and/or diffusible contrast agents to the subject for radiological imaging.

U.S. Patent Application Publication No. 20200353104 to Markus Berger at al. disclosed a new class of high relaxivity extracellular gadolinium chelate complexes (i.e., tetragadolinium) as MRI contrast agents.

U.S. Patent Application Publication No. 20200325108 to Luciano Lattuada et al. disclosed a new class of functionalized macrocycles capable of chelating paramagnetic metal ions, their chelated complexes with metal ions and as MRI contrast agents.

U.S. Patent Application Publication No. 20210000983 to Kwang Yeol Lee et al. disclosed a metal oxide nanoparticle-based T1-T2 dual-mode MRI contrast agent. The dual-mode MRI contrast agent can provide more accurate and detailed information associated with disease than single MRI contrast agent in both T1 imaging with high tissue resolution and T2 imaging with high feasibility on detection of a lesion.

WO2021043926 to Brian C Bales et al. disclosed isomers of Manganese chelate compounds as MRI contrast agents.

The paper “Blood-brain barrier permeation: molecular parameters governing passive diffusion” in The Journal of membrane biology. 1998. 165 (3): p. 201-211 to Fischer H et. al reviews multiple modification methods for enhancing their penetrations across the BBB that include pegylation, esterification, addition of fatty acids, insertion of d-amino acids, reversal of their primary amino acid sequence, production of nanoparticles, and glycosylation with glucose or other sugars.

The paper “trans-Sodium Crocetinate and Diffusion Enhancement” in The Journal of Physical Chemistry B. 2006; 37: p. 18078-18080 to Amanda K. Stennett et al. discloses that Trans-Sodium crocetinate (TSC) increases the diffusion coefficient of glucose through water by about 25-30% using increased hydrogen bonding of the water molecules, and molecular simulations suggest that the increase in diffusivity occurs only in these ordered regions.

Contrast agents can be used to highlight specific structures to improve visualization of living and deceased organisms and lesions. Current contrast agents used for medical imaging focused on the properties of T₁, T₂ or T₂*, and CEST in MR imaging; absorption rate for CT and x-ray; and reflection of the ultrasound waves for ultrasound tomography, after the administration of the contrast agents. Most of these contrast agents are extremely unstable and/or toxic in biological conditions. These compounds hold a potential safety risk for some patients and this has spurred research into alternatives. Therefore, there remains an unmet need for safe and effective contrast agents in medical imaging, and for characterization of contrast agent transport across BTB in vivo.

This background information is provided for the purpose of making known information believed by the applicant to be of possible relevance to the present invention. No admission is necessarily intended, nor should be construed, that any of the preceding information constitutes prior art against the present invention.

### SUMMARY

This disclosure describes a type of new contrast agents which can be used to enhance the diffusivity contrast in a subject after the administration of the new contrast agents herein. In this invention, the diffusivity contrast agents, for the first time, are proposed to enhance the diffusivity contrast in most medical imaging modalities (e.g. ultrasound, MRI. CT, PET, SPECT, optical imaging). The proposed diffusivity contrast agents can be obtained not only from nanoparticles, polymers, compounds, but also directly from foods, nutrient substances and drugs. Herein the proposed diffusivity contrast agents may directly be selected from the FDA approved substances, therefore reducing the money and time costs for developmental and regulatory procedures as well as minimizing contrast agent administration risks.

It should be understood that this disclosure contemplates using MRI, which is provided as an example only, with the techniques described herein. Alternatively or additionally, the transport of a substance across BTB after the administration of the substance, is detected by at least one of, a magnetic resonance imaging (MRI) apparatus, a positron emission tomography (PET) apparatus, a computed tomography (CT) apparatus, ultrasound tomography, optical imaging, and a single positron emission computed tomography (SPECT) apparatus.

An example method for medical imaging using an diffusivity contrast agent comprises: obtaining the diffusivity contrast agent having specific diffusivity, wherein the diffusivity contrast agent is configured to pass the BTB; administering a detectable dose of the diffusivity contrast agent to a subject; acquiring one or more post-contrast images for a region of interest after the administration of the contrast agent, wherein the post-contrast images include changes in diffusivity of the region of interest compared to one or more images acquired without said diffusivity contrast agent; and characterizing a transport of the diffusivity contrast agent in the region of interest based on the changes in diffusivity of the region of interest.

Alternatively or additionally, in some implementations, the new diffusivity contrast agent as used herein is obtained based on the diffusivity of the diffusivity contrast agent in targeted human tissues or lesions. In some implementations, the new diffusivity contrast agent may be obtained by modulating the diffusivity of substances to obtain the diffusivity contrast agent having a detectable diffusivity on targeted tissues or lesions of a subject. In some implementation, the new diffusivity contrast agent may be obtained by synthesizing the diffusivity contrast agent using substances, the diffusivity contrast agent having a detectable diffusivity on targeted tissues or lesions of the subject.

Alternatively or additionally, in some implementations, diffusivity contrast agents are designed, manufactured, characterized and used according to their diffusivity in targeted human tissues or lesions, and BTB permeability.

Alternatively or additionally, in some implementations, the contrast agent passes BTB via one or more of passive diffusion transport pathway, paracellular transport pathway, carrier-mediated transport pathway, receptor-mediated transcytosis pathway, adsorptive-mediated transcytosis pathway, and cell-mediated transport pathway.

Alternatively or additionally, in some implementations, the diffusivity of the new contrast agent can be modulated by at least one of physical modifications, chemical modifications, other methods and their variations.

Alternatively or additionally, in some implementations, the detectable transport of administrated diffusivity contrast agents can be further applied for understanding and monitoring a drug delivery when the drug is used as a diffusivity contrast agent.

It should be understood that the above-described embodiments and aspects may also be described and illustrated in conjunction with systems, compositions, articles of manufacture, and methods which are meant to be exemplary and illustrative, not limiting in scope.

Other systems, methods, features and/or advantages will be or may become apparent to one with skill in the art upon examination of the following drawings and detailed descriptions. It is intended that all additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flowchart illustrating a method for medical imaging using a diffusivity contrast agent as a contrast agent.

FIG. 2 is a flowchart illustrating example methods for obtaining diffusivity contrast agents.

FIG. 3 is a flowchart method illustrating example operations for modulating the transport of diffusivity contrast agents across biological barriers.

FIG. 4 is a flowchart method illustrating example methods for modulating the diffusivity of diffusivity contrast agents.

### DETAILED DESCRIPTION

### 1. Definition

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not.

The term “subject” as used herein refers to a “human”, including man, woman, child and human at the prenatal stage. Additionally, a subject may be a “patient” who is under health care. The patient may be waiting for this care or may be receiving it or may have already received it.

The term “biological membrane, biomembrane or cell membrane” as used as herein refers to a selectively permeable membrane which separates cell from the external environment or creates intracellular compartments.

The terms “contrast materials”, “contrast agents” and “contrast media” as used interchangeably herein, refers to a substance that is physiologically tolerable and capable of to increase the contrast or improve more information of structures or fluids within the body in medical imaging. They can be used to diagnose disease as well as monitor treatment effects. The contrast agent is administered by oral or intravenous administration. A suitable contrast agent is preferably biocompatible, e.g., non-toxic, chemically stable, not absorbed by the body or reactive with a tissue, and eliminated from the body within a short time. For example, MRI contrast agents are used to improve the visibility of internal body structures. The most commonly used compounds for contrast enhancement are gadolinium-based which shorten the relaxation times (e.g., T₁ or T₂ relaxation times) following oral or intravenous administration. The disadvantage of exogenous tracers is that there are side effects associated with the administration of the tracers. For example, the injection of exogenous agent of Gd-based contrast agent in MRI has potential side effects of nephrogenic systemic fibrosis (NSF), deposition of Gd molecules, and potential neurotoxicity.

The term “blood tissue barrier” as used herein include at least one of blood-brain barrier, but not limit to, blood-cerebrospinal fluid (CSF) barrier, the blood-retinal barrier, the blood-testis barrier. The term “blood brain barrier (BBB)” as used herein refers to a highly selective border that separates the circulating blood from the brain. The blood-brain barrier is composed of endothelial cells of the capillary wall, astrocyte end-feet ensheathing the capillary, and pericytes embedded in the capillary basement membrane. BBB greatly restrict exchange (e.g. influx/efflux) of substances between capillaries and brain tissue. The term “blood-CSF barrier” as used herein is an obstruction or a partition separating the blood from CSF. The term “blood-retinal barrier” consists of cells that are joined tightly together to prevent certain substances from entering the tissue of the retina. Diabetic retinopathy is related to the breakdown of the blood-retinal barrier.

The terms “therapy” and “treatment” as used interchangeably herein, refer to an intervention performed with the intention of improving a subject's status.

The terms “detection” and “diagnosis” as used interchangeably herein, refer to identify the abnormal tissue or lesion.

The terms “brain” and “head” as used herein may designate all or part of the cerebral lobes (i.e., frontal lobe, parietal lobe and occipital lobe), including the cerebral cortex.

The term “bio-dissolvable or biodegradable substance” as used herein refers to a substance which over time is partially or fully absorbed by the body. Such materials can include gels and/or hydrogels, polymers, or other suitable substance s. Such substance can be synthetic, naturally occurring, or a blends or composites thereof.

The term “drug” as used herein is defined as a medicament or medicine which is used for the therapeutic treatment of a medical condition or disease. The drug may be used in combination with another drug or type of therapy.

The term “compound” as used herein may a substance formed when two or more chemical elements are chemically bonded together, for example, metal oxide compounds and Gd based chelates.

The term “polymer” as used herein refers to any compound that is made up of two or more monomeric units covalently bonded to each other, where the monomeric units may be the same or different. For example, Abilify, Nexium. Unlike traditional small molecule substances, polymer is a macromolecule that its repeating unit nature allows for easy incorporation of multivalent features on size scales not accessible to small molecules.

The term “substituted” as used herein refers to a molecule or functional group in which one or more hydrogen atoms have been replaced by other atom(s) or group(s) selected from the group of substituents. Examples of substituents include, but are not limited to, carbon-based substituents, oxygen-based substituents, nitrogen-based substituents, sulfur-based substituents and their combinations thereof.

The term “natural product” as used herein refers to a chemical compound or substance produced by a living organism. The natural product includes any substance produced by life. For acquisition of crude natural product materials from terrestrial and marine environments for extraction and screening of extracts in the NCI 60 cancer cell line screen and other intramural and extramural screening programs [https://dtp.cancer.gov/organization/npb/default.htm].

The term “administration” and its variant as used herein refers to introducing a substance into a subject. In some embodiments, a route of administration is oral administration or intravenous administration. However, any route of administration, such as topical, subcutaneous, peritoneal, intraarterial, inhalation, vaginal, rectal, nasal, introduction into the cerebrospinal fluid, or instillation into body compartments can be used.

The term “subject” as used herein includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

By “administration” is meant introducing a compound into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

The term “nanoparticle” as used herein refers to particles having a particle size on the nanometer scale, less than 0.5 micrometer.

The term “lipophilicity” as used herein is defined as the logarithm of the ratio of a substance that partitions into organic phase to that in aqueous phase, and is referred as log P.

The term “variant” as used herein refers to a compound or a polymer that differs from a reference a compound or a polymer, but retains essential properties. A variant of a compound or a polymer can be modified by substitutions, additions, and/or deletions of the chemical elements. For example, new contrast agents may be variants of existing substances that can pass the blood-tissue barriers, but their diffusivity is modulated. The variant may be naturally occurring, or it may be a variant that is not known to occur naturally.

The term “diffusivity contrast agent” as used herein is defined as a drug, compound, nanoparticle, polymer, composition, biological entity or their variant which is used as a contrast agent in medical imaging, wherein medical imaging can characterize the diffusivity qualitatively and/or quantitatively.

### 2. Diffusivity in Liquids

As for a spherical molecule, its diffusion coefficient in a quiescent fluid at uniform temperature is given by Stokes-Einstein equation:

$$D = \frac{{k}_{B} T}{6 πη r} \qquad Equation ( 1 )$$

where solvent viscosity η and solution radius r of the molecule moving with uniform velocity.

As for a small molecule, its diffusion coefficients in liquids is generally given by Wilke-Chang equation:

$$D = \frac{1.173 \times 1 {0}^{- 10} {( ϕ M )}^{0.5} T}{η {V}_{m}^{0.6}} \qquad Equation ( 2 )$$

where D is liquid diffusivity (meter² second⁻¹), ϕ is an association factor for the solvent (2.6 for water, 1.9 for methanol, 1.5 for ethanol, 1.0 for unassociated solvents), M is molecular mass of solvent, η is viscosity of solvent (pascal·second), T is absolute temperature (Kelvin), and Vₘ is molar volume of the solute at its boiling point (meter³/kmol). For example, the viscosity of blood at 37° C. is normally 3×10⁻³ to 4×10⁻³ (pascal·second); the viscosity of human brain tissue at 37° C. is around 3.4×10⁻³ (pascal·second) [3]; and the viscosity of water at 37° C. is around 0.69×10⁻³ (pascal·second).

If the sample is a mixture containing N components, the molar volume is approximated as, using the density of the mixture:

$${V}_{m} = \frac{{\sum }_{i}^{N} {x}_{i} {M}_{i}}{{ρ}_{m i x}} \qquad Equation ( 3 )$$

where xᵢ and Mᵢ are the mole fraction and the molar volume of its individual component i, respectively. ρₘᵢₓ is the density of the mixture.

According to Equations (2) and (3), the major methods to modulate diffusivity of a given substance in human tissue include: 1) altering the mole fractions of solvent (e.g., increased permeability of solvent cross BTB); 2) modifying mass weight by substitutions, additions, and/or deletions; 3) altering viscosity of solution.

### 3. Overview

Any variation in the structure and function of BTB due to physical changes, environmental factors, toxins, infection, mutation, aging, etc., may result in various central nervous system (CNS) disorders, such as Alzheimer's disease, brain cancer, multiple sclerosis, Parkinson's disease and stroke. In 2019, it was reported that nearly 1.5 billion people suffer from various CNS disorders across the globe. In fact, the incidence of CNS disorders is anticipated to increase by 12% by 2030. These diseases always associate with BTB dysfunction. The investigation about the transport of substance across BTB is very important for the diagnosis and treatment of these diseases.

The use of contrast agents in medical imaging to increase the contrast of structures or fluids within the human body in medical imaging has a long history. There are millions of radiological exams with contrast agents worldwide and the global contrast agent market size is above 5.0 billion US dollar annually. The development of new safe and effective contrast agents is an unmet need in medical imaging. Ideally, contrast agents should accumulate with a sufficient concentration at the pathological site, ensuring specific diagnosis and assess the treatment. Most of the contrast agents comprise small molecules accumulating at pathological sites due to BTB. For example, most current MRI contrast agents, such as Gd-based chelates, are either paramagnetic metal ion-ligand complexes or superparamagnetic particles with decreased T₁ and T₂ relaxation time of water protons. Iodine-based and barium-sulfate compounds are used as contrast agents in x-ray and CT exams; microbubbles and microspheres are used as contrast agents in ultrasound imaging exams. But these FDA approved contrast agents still have potential risk for some patient population. For example, all MRI contrast agents currently used in the clinic have been synthetic, with most based on paramagnetic metal complexes, such as Gd³⁺ chelates. Gd-based contrast agents are contraindicated for patients with acute kidney injury or severe chronic kidney disease. These risks have led to renewed interest in finding alternatives to these contrast agents. However, the translation of new contrast agents to actual clinical application is time-consuming and expensive. It will save a lot of time and money if the new diffusivity contrast agents can be selected based on the FDA approved substances with minimal potential risks, for example, D-glucose and caffeine.

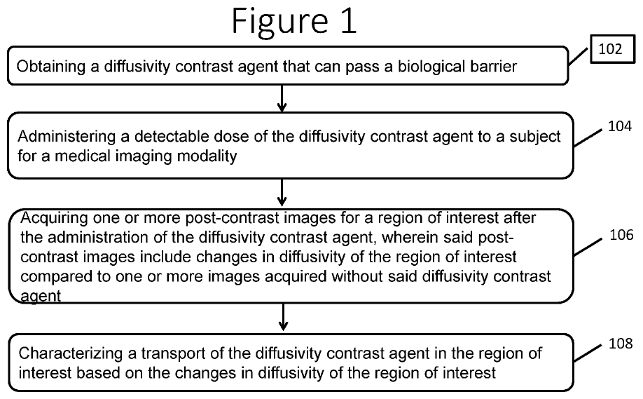

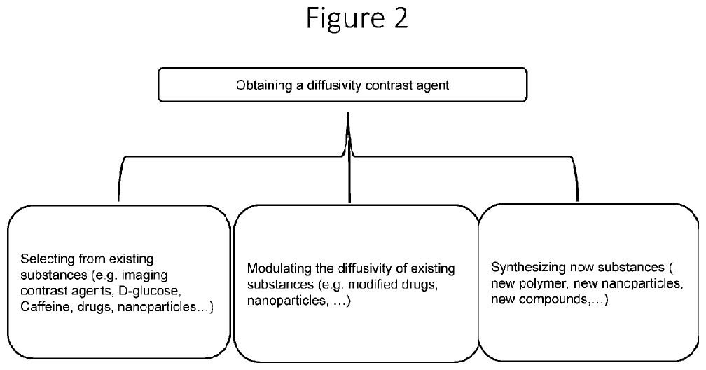

FIG. 1 shows a flowchart illustrating a method for medical imaging using an example diffusivity contrast agent as a contrast agent. In step 102, the method obtains a diffusivity contrast agent as an exogenous contrast agent, wherein the diffusivity contrast agent has specific diffusivity or viscosity and can pass the blood-tissue barrier. For example, the diffusivity of the diffusivity contrast agent may be about 10⁻⁸ meter² second⁻¹ to 10⁻⁹ meter² second⁻¹. As another example, the diffusivity of the diffusivity contrast agent may be about 10⁻⁹ meter² second⁻¹ to 10⁻¹⁰ meter² second⁻¹. As another example, the diffusivity of the diffusivity contrast agent may be about 10⁻¹⁰ meter² second⁻¹ to 10⁻¹¹ meter² second⁻¹. In embodiments, the viscosity of the diffusivity contrast agent in blood may be about 1×10⁻⁴ pascal·second to 1×10⁻³ pascal·second. Various ways of obtaining a diffusivity contrast agent will be described further in detail with reference to FIG. 2 below.

In step 104, the method administers a detectable dose of the diffusivity contrast agent for at least one medical imaging modality to a subject. The medical imaging modality includes one or more of MRI, PET, SPECT, CT, x-ray, optical imaging and ultrasound.

In step 106, the method acquires one or more post-contrast images for a region of interest after the administration the diffusivity contrast agent. The post-contrast images include changes in diffusivity of the region of interest compared to one or more images acquired without said diffusivity contrast agent.

In step 108, the method characterizes a transport of the diffusivity contrast agent in the region of interest based on the changes in diffusivity of the region of interest.

In this invention, the proposed diffusivity contrast agents are designed ideally following the requirements of clinical applications: The diffusivity contrast agents should 1) improve the visualization of the target tissues or lesion by the difference in diffusivity with and without the contrast agents; 2) have small molecules so that they have a high permeability of BTB. The mass weight should be less than 2000 Dalton; 3) have sufficiently long tissue retention-time (e.g., 10-200 minutes) for completion of medical imaging procedures; 4) localize or target the regions of interest and possess favorable biodistribution and pharmacokinetic profiles; 5) be readily soluble or form stable suspensions at aqueous physiological conditions (appropriate pH and osmolality) with low viscosity; 6) be non-toxic and can be cleared from the body in a reasonably short amount of time (<24 h).

Alternatively or additionally, in some embodiments, the diffusivity contrast agents can be administered to the patient orally, rectally, or intravenously

FIG. 2 shows a flowchart illustrating example operations for obtaining diffusivity contrast agent. The operations include: selecting contrast agents from substances according to the diffusivity of the substances in targeted human tissues or lesions; or modulating the diffusivity of substances to obtain the diffusivity contrast agent having a detectable diffusivity on the targeted human tissues or lesions; or synthesizing the diffusivity contrast agent using substances, which has a detectable diffusivity on the targeted human tissues or lesions.

Alternatively or additionally, in some embodiments, the diffusivity contrast agents can be selected from existing FDA approved contrast agents, drugs, nano-particles, food, or nutrient substances.

The diffusivity contrast agent may be selected from contrast agents comprising:
MRI contrast agents and their variants, such as gadopentetate dimeglumine, Gadobutrol, Gadoterate meglumine, Gadoteridol injection, Ferric ammonium citrate, Manganese Chloride, Ferristene (MPIO), Ferumoxides (SPIO). Most MRI contrast agents such as Gd-based MRI contrast, Mn-based MRI contrast agent, are metal chelation, while diffusivity contrast agents can be non-metal. Though most contrast agent used in MRI are paramagnetic or super-paramagnetic, the diffusivity contrast agents herein may not be paramagnetic or super-paramagnetic.
CT contrast agents, such as Ethiodized oil, Ioversol, Iohexol, Iopromide, Iodixanol, Ioxaglate, Iothalamate, Iomeprol, Ioxilan, Ioxaglate, and Iopamidol.
Ultra-sound contrast agents and their variants, such as Albunex, Levovist, Optison and Sonazoid, and Lunason.
PET contrast agents, such as Fluodeoxyglucoss

Conventional contrast agents are not specifically designed based on their diffusivity contrast agents. For example, most MRI contrast agents are designed based on their magnetic properties of contrast agents (e.g. paramagnetic or superparamagnetic properties) that modulate their T₁ and T₂ relaxation time. A lot of conventional contrast agents are either paramagnetic or superparamagnetic. In contrast, the present disclosure characterizes a transport of the diffusivity contrast agent based on their diffusivities. The diffusivity contrast agent is a novel contrast agent because the diffusivity contrast agents have new physicochemical property (i.e., diffusivity) that totally differ from the physicochemical property of conventional contrast agents (such as T₁ and T₂ relaxation time). Diffusion is the most common phenomenon in the living world and the diffusivity is an important physicochemical property of molecules. The use of diffusivity of contrast agents is generally not considered to enhance the image contrast in standard practice. The present disclosure obtains diffusivity contrast agents as new contrast agents according to their diffusivities.

The proposed diffusivity contrast agents can be selected directly from safe and natural food or nutrient substances, for example, water-soluble or lipid-soluble food ingredients or nutrients or dietary supplements comprising at least one of caffeine, sucrose, glucose, amino acids, lactate, pyruvate, glutamine, folates, fatty acid, ascorbic acid, water-soluble vitamins, peptides, inositol, riboflavin, thiamine, thiamine monophosphate, niacin, pyridoxine, pyridoxal phosphate, pantothenic acid, biotin, lipoic acid, nucleosides, purine bases, proteins and their variants, wherein the selected diffusivity contrast agents can pass BTB to entering human tissues. More potential diffusivity contrast agents can be selected form the United States Pharmacopoeia.

In some embodiments, the proposed diffusivity contrast agents are selected from the group consisting of polyethylene glycol, but not limited to, 2-methyl-2-oxazoline, 2-ethyl-2-oxazoline, 2-isopropyl-2-oxazoline, acrylamide, methacrylamide, vinyl alcohol, hydroxyethyl acrylate, hydroxyethyl methacrylate, phosphate, citrate, sulfosalicylate, and acetate and their variants. More potential diffusivity contrast agents can also be selected from the United States Pharmacopoeia.

In some embodiments, diffusivity contrast agents are selected from metals, metal ions, polymers, peptides, nucleotides, saccharides, ligands, lipids, proteins, chelates, antibodies, nanoparticles, liposomes, or their variants.

Additionally, diffusivity contrast agents are selected from the group consisting of aspartic acid, but not limited to, glutamic acid, methacrylic acid, acrylic acid, malic acid and their variants.

Lipinski's “rule of five” relates BTB permeability to molecular weight, lipophilicity, polar surface area, hydrogen bonding, and charge: There are less than 5H-bond donors (expressed as the sum of nitrogen-hydrogen and oxygen-hydrogen bonds); The molecular weight is over 400; The lipophilicity Log P is less than 5; There are less than 7H-bond acceptors (expressed as the sum of nitrogen and oxygen bonds). However, it is well known that compounds which violate these rules can still be very successful as drugs. Herein the diffusivity contrast agents are preferably selected from water-soluble or lipid-soluble drugs that can enter tissues across BTB comprising at least one of temozolomide, trifluoperazine quinacrine, and their variants. The diffusivity contrast agents can be selected from the FDA approved drugs, such as temozolomide, carmustine (BCNU), lomustine (CCNU), and platinum drugs. More potential diffusivity contrast agents can be obtained from the public drugbank database: https://www.drugbank.ca/drugs. In summary, a drug used as a therapeutic agent can also be used as a diffusivity contrast agent in medical imaging based on the physical and chemical properties of the drug, including diffusivity, but not limited to, density, electromagnetic property, chemical exchange, relaxation time.

Optionally, a drug used as a diffusivity contrast agent may be used as a tool for real-time monitoring of drug delivery, enabling a personalized and optimized approach to drug administration in individual patients.

Optionally, the diffusivity contrast agents are selected from nanoparticles which can pass BTB. The nanosized contrast agent ranges from 1 to 200 nm, including, but not limit to, MRI nano-particle contrast agents, CT nano-particle contrast agents, ultra-sound nano-particle contrast agents, PET nano-particle tracers, metal nano-particle, metal oxide nano-particle and their variants. For example, the diffusivity contrast agents can be selected from nanosized molecules, such as iron oxide nanoparticle, manganese oxide nanoparticle, gadolinium oxide nanoparticle, other metal oxide nanoparticle, gold nanoparticle, iohexol, iopentol, iopamidol, iodixanol, iopromide, iotrolan, metrizamide, poly amidoamines dendrimers, polypropylene imine, Yttrium nitrate hexahydrate, Ethylene glycol, and their variants.

Though the diffusivity contrast agents are designed herein as a passive diffusion pathway to pass contrast agents to targeted sites across BTB, other five main pathways, such as paracellular transport, carrier-mediated transport, receptor-mediated transcytosis, adsorptive-mediated transcytosis, and cell-mediated transport, can also transport the contrast agents to targeted sites. According to the different pathways, the diffusivity contrast agents can be designed and manufactured. The two major concerns about the diffusivity contrast agents are 1) ability to delivery across BTB and 2) detectable diffusivity changes.

Optionally, the diffusivity contrast agent can be a polymer, such as peptides, polysaccharides, nucleic acids and the like, where the polymers may be naturally occurring or synthetic.

Optionally, the mass weight of the diffusivity contrast agents are about 1 to 100 Daltons; or about 100 to 800 Daltons; or about 800 to 6000 Daltons.

Though the exogenous diffusivity contrast agents are mainly discussed herein, the endogenous diffusivity contrast agents should be available. Various experiments indicated that pathological changes lead to the diffusivity changes, which can be used as an endogenous contrast agent, like blood in blood oxygenation level dependent imaging.

In an embodiment, images are generated after the administration of diffusivity contrast agents for various medical imaging modalities, such as an MRI apparatus, a PET apparatus, a CT apparatus, and a SPECT apparatus, an ultrasound tomography apparatus, and combinations thereof.

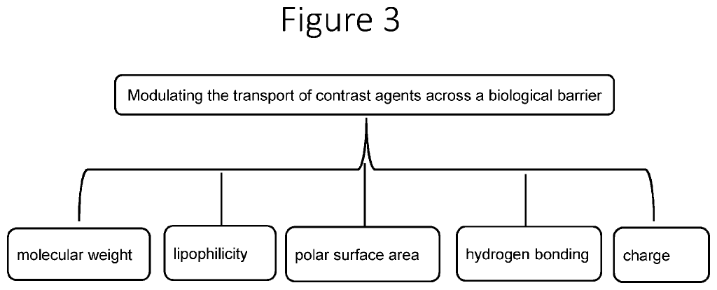

FIG. 3 shows a flowchart method illustrating example operation for modulating the transport of diffusivity contrast agents across BTB. The method includes modulating one or more mass weight, lipophilicity, polar surface area, hydrogen bonding, and charge of the contrast agents.

All selected diffusivity contrast agents enable to pass BTB. Some studies indicate that a substances with superior BBB/BBTB permeability should have the following properties: the substances possess appropriate lipid-solubility, which can be estimated by the count of aromatic rings and/or alicyclic rings; the substances are structurally featured with the trialkylamine-based moiety, including dimethylamine and a series of nitrogen-containing heterocycles such as piperidine, piperazine, and morpholine; the substances can traverse the BBB/BBTB based on the Lipinski's “rule of five”. For example, small molecule drugs may cross the BBB via lipid-mediated free diffusion, providing the drugs have a molecular weight <400 Da and forms <8 hydrogen bonds. The contrast agent permeability across the BBB is also favored by drugs' lipophilicity, low molecular weight and lack of ionization at physiological pH. For example, the permeability of very lipid-soluble compounds includes ethanol, nicotine, iodoantipyrine and diazepam. The permeability can be modulated by the addition of hydrophobic groups to the contrast agent to improve its diffusivity. For example, the addition of methyl groups to various drugs can increase lipophilicity and brain penetration. Therefore, the addition of specific molecules can modulate the lipidization and BTB permeability of the selected contrast agents. It is noted that the added molecules will increase the molecular sizes of the selected contrast agents and the increased sizes of the contrast agents influence BBB permeability decreases exponentially. The increased lipidization of the diffusivity contrast agents will simultaneously increase influx and efflux across BTB, leading to short tissue retention-time. In a word, the design and manufacture of the diffusivity contrast agents should consider the trade-off of various factors, such as lipidozation and mass weight, to optimize the BTB permeability and diffusivity of contrast agents in human tissue.

Like prodrug approach, a pro-agent corresponding to a diffusivity contrast agent can be used to improve the BTB permeability and the diffusivity of the contrast agent. The pro-contrast agent approach can include the coupling of the active contrast agents with, but not limited to, lipid moieties, such as fatty acids, glycerides or phospholipids.

Though passive diffusion transport is the most common way to enhance the permeability of contrast agents across BTB, several strategies, such as adsorptive-mediated transcytosis, carrier-mediated transport, receptor-mediated transport, active efflux transport and peptide vector strategies, can be used to enhance the permeability. For example, the carrier-mediated transport is mediated by a series of the solute carrier transporters. It can deliver some specific substances such as sugars, amino acids, organic cations and anions, nutrients and metabolites into the brain. Additionally, receptor-mediated transport is a specific transcytosis mechanism based on endocytosis from the luminal side and exocytosis from the abluminal side of the endothelium. It can deliver some macromolecules into the brain.

Additionally, ultra-sound and electromagnetic field can be implemented to open the BTB barrier and improve the permeability of the diffusivity contrast agent.

The diffusivity contrast agent is used to improve contrast in medical imaging based on the diffusivity difference between the contrast agent and around human tissue. Various methods can be used to improve the diffusivity of the contrast agent according to Equations 1-3. For example, WO2009058399A1 and US20130018014A1 to John L. Gainer disclosed a method to enhance the diffusivity of the aqueous system through increased specific volume by affecting the extent and strength of hydrogen bonding among water molecules. The paper “Blood-brain barrier permeation: molecular parameters governing passive diffusion” in The Journal of membrane biology. 1998. 165 (3): p. 201-211 to Fischer H et. al reviews multiple modification methods for enhancing their penetrations across the BBB that include pegylation, esterification, addition of fatty acids, insertion of d-amino acids, reversal of their primary amino acid sequence, production of nanoparticles, and glycosylation with glucose or other sugars. The paper “trans-Sodium Crocetinate and Diffusion Enhancement” in The Journal of Physical Chemistry B. 2006; 37: p. 18078-18080 to Amanda K. Stennett et al. discloses that Trans-Sodium crocetinate (TSC) increases the diffusion coefficient of glucose through water by about 25-30% using increased hydrogen bonding of the water molecules, and molecular simulations suggest that the increase in diffusivity occurs only in these ordered regions.

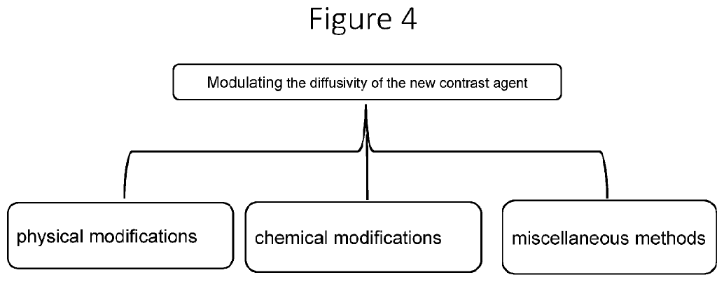

FIG. 4 shows a flowchart illustrating example operations for modulating the diffusivity of the contrast agent. The diffusivity improvement techniques for the diffusivity contrast agents can be categorized into physical modification, chemical modifications of the drug substance, and other techniques.

Physical modifications are generally carried out by using simple, inexpensive and safe physical methods because they require no chemicals or biological agents. The methods of physical modifications include, but not limited to, particle size reduction of solvent using micronization (e.g., vacuum ball mill) and nanosuspension technology, mechanical activation with stirring ball mill; pulse dielectric fields treatment, corona electrical discharges, and their combinations thereof. Physical modifications modify the diffusivity of the contrast agent in human tissue through the modulation of contrast agent solvent size, shape, and viscosity of the contrast agent using physical methods.

Chemical modifications are carried out by the modification, addition or removal of the contrast agent through chemical reaction. The methods of chemical modification include, but not limited to, modulation of PH, use of buffer, derivatization, complexation, salt formation, and their combinations thereof. Poor water-soluble contrast agent may dissolve in water by a pH modulation. Additionally, mass weight of contrast agent can be modified by addition or removal of some chemical elements and then the diffusivity of the contrast agent can be modulated. For example, the hydrogen on the benzene ring can be substituted by chlorine, fluorine, methoxyl group, and trifluoromethyl group.

In some embodiments, one or more of the hydrogen atoms of a ring may be optionally substituted by a substituent. The substituent is at least one of carbon-based substituent, oxygen-based substituent, nitrogen-based substituent, sulfur-based substituent, phosphate-based substituent, and their combinations thereof.

In some embodiments, the ring or rings may be monocyclic, bicyclic, polycyclic, spirocyclic, fused, bridged, or linked.

In some embodiments, pH and contrast agent concentration can modulate the diffusivity of contrast agents, such as proteins.

Other methods are also proposed to modulate the solubility of diffusivity contrast agents in blood and/or the diffusivity of the contrast agents. The methods include, but not limited to, supercritical fluid process, surfactant, solubilizers, cosolvency, hydrotrophy, and novel excipients. For example, surfactant can be used to reduce surface tension and improve the dissolution of lipophilic contrast agent in blood. Commonly used non-ionic surfactants are lauroyl macroglycerides, castor oil, di-fatty acid ester of low molecular weight polyethylene glycol. Hydrotrophy may enhance the solubility of contrast agent by using sodium benzoate, urea, sodium citrate, and sodium salicylate.

Alternatively or additionally, in some implementations, modulating the diffusivity of the selected or synthesized substances (e.g., increasing or decreasing the diffusivity of the substances) are performed by one or more operations of altering concentration of the contrast agent; adjusting mass weight by replacing one of element with other element; increasing solubility; modifying viscosity of the contrast agent by mixture with other solution and compound; and altering temperature of the contrast agent.

Alternatively or additionally, modulating the diffusivity of the contrast agent includes one or more procedures of physical modifications, chemical modifications, other methods, and their variations.

Alternatively or additionally, in some implementations, modifying the diffusivity of substances is comprised by modulation in one or more of specific volume of the solute, mass weight of solvent, viscosity of solvent, and concentration.

Alternatively or additionally, in some implementations, the use of a diffusivity contrast agent with different properties is as a contrast agent in one or more imaging modalities. For example, T₁ relaxation and diffusivity of Gd-based contrast agent may enhance T₁-weighted MRI imaging contrast and diffusion weighted MRI contrast respectively.

Alternatively or additionally, in some implementations, the modulating the diffusivity of existing substances to obtain a detectable diffusivity on the targeted human tissues or lesions can be carried out by 1) converting water-soluble drugs that do not penetrate the BBB into lipid-soluble drugs that do cross the BBB, 2) concentration gradient of both solute and contrast agents in a multicomponent diffusion system, or 3) changing the specific volume via physical and chemical modifications.

Alternatively or additionally, in some implementations, the diffusivity contrast agent can optionally be either a magnetic compound or a non-magnetic compound. For example, D-glucose used as a diffusivity contrast agent is a non-magnetic compound. But Gd-based contrast agent is a magnetic compound.

Alternatively or additionally, in some implementations, the diffusivity contrast agent is either metal or non-metal. Alternatively or additionally, in some implementations, the diffusivity contrast agent is either organic or inorganic. Alternatively or additionally, in some implementations, the diffusivity contrast agent is non-paramagnetic and non-super-paramagnetic. Alternatively or additionally, in some implementations, the diffusivity contrast agent is a non-specific distribution across entire human body.

Alternatively or additionally, the diffusivity contrast agent is endogenous contrast agent which has lower toxicity than drugs and exogenous contrast media, for example, de-oxygenhemoglobin in blood.

Alternatively or additionally, the diffusivity contrast agent may be non-specific agent for body compartment, cell, organ, or tissue. Alternatively, the agent may be targeted agent that has a specific affinity for a particular body compartment, cell, organ, or tissue.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide some, procedural or other details supplementary to those set forth herein before and after intravenous ethanol administration

Increased diffusion coefficient may lead to a positive diffusion contrast agent. In contrary, decreased may lead to a negative diffusion contrast agent. MRI contrast agent changed with different static field strength. But the diffusion contrast agent is independent of static field strength.

What is claimed:

1. A method for medical imaging using a diffusivity contrast agent used as an exogenous contrast agent, the method comprising:
obtaining the diffusivity contrast agent having specific diffusivity, wherein the diffusivity contrast agent is configured to pass a biological barrier of a subject;
administering to the subject a detectable dose of the diffusivity contrast agent for at least one medical imaging modality, where the medical imaging modality includes one or more of MRI, PET, SPECT, CT, x-ray, optical imaging and ultrasound;
acquiring one or more post-contrast images for a region of interest after the administration of the diffusivity contrast agent, wherein said post-contrast images include changes in diffusivity of the region of interest compared to one or more images acquired without said diffusivity contrast agent; and
characterizing a transport of the diffusivity contrast agent in the region of interest based on the changes in diffusivity of the region of interest.

2. The method of claim 1, wherein the biological barrier includes at least one of a cell membrane, a nuclear membrane, a blood-tissue barrier, and mucosal membranes.

3. The method of claim 1, wherein obtaining the diffusivity contrast agent further comprises:
selecting the diffusivity contrast agent based on the diffusivity of the diffusivity contrast agent in targeted human tissues or lesions.

4. The method of claim 1, wherein obtaining the diffusivity contrast agent further comprises:
modulating diffusivity of substances to obtain the diffusivity contrast agent having a detectable diffusivity on targeted tissues or lesions of the subject.

5. The method of claim 1, wherein obtaining the diffusivity contrast agent further comprises:
synthesizing new substances, the diffusivity contrast agent having a detectable diffusivity on targeted tissues or lesions of the subject.

6. The method of claim 3, wherein the diffusivity contrast agent is selected from one or more of metals, metal ions, polymers, peptides, nucleotides, saccharides, ligands, lipids, proteins, chelates, antibodies, nanoparticles, liposomes, or their variants.

7. The method of claim 3, wherein the diffusivity contrast agent is selected from a drug, a protein, a peptide, an antibody, an antibody fragment, a ligand, a cytokine, an inhibitory substance, a stimulatory substance, a nanoparticle, a nutrient substance, a food, a compound, a drug and their variants thereof.

8. The method of claim 4, further comprising modulating the diffusivity of the substances by one or more operations of altering concentration of the substances; adjusting mass weight of the substances by replacing one element with another element; increasing solubility of the substances; modifying viscosity of the substances by mixture with other solution and compound; and altering temperature of the substances.

9. The method of claim 5, further comprising synthesizing the diffusivity contrast agent using substances by one or more operations of altering concentration of the substances; adjusting mass weight of the substances by replacing one element with another element; increasing solubility of the substances; modifying viscosity of the substances by mixture with other solution and compound; and altering temperature of the substances.

10. The method of claim 4, wherein modulating the diffusivity of the substances includes one or more procedures of physical modifications or chemical modifications to the substances.

11. The method of claim 4, wherein modulating the diffusivity of the substances comprises modulation in one or more of specific volume of a solute including the substances, mass weight of a solvent including the substances, and viscosity of the solvent including the substances.

12. The method of claim 1, wherein the diffusivity contrast agent is neither paramagnetic nor super-paramagnetic substances.

13. The method of claim 1, wherein mass weight of the diffusivity contrast agent is about 1 Daltons to 100 Daltons.

14. The method of claim 1, wherein mass weight of the diffusivity contrast agent is about 100 Daltons to 800 Daltons.

15. The method of claim 1, wherein mass weight of the diffusivity contrast agent is about 800 Daltons to 6000 Daltons.

16. The method of claim 1, wherein the diffusivity of the diffusivity contrast agent is about 10⁻⁸ meter² second⁻¹ to 10⁹ meter² second⁻¹.

17. The method of claim 1, wherein the diffusivity of the diffusivity contrast agent is about 10⁻⁹ meter² second to 10⁻¹⁰ meter² second⁻¹.

18. The method of claim 1, wherein the diffusivity of the diffusivity contrast agent is about 10⁻¹⁰ meter² second⁻¹ to 10⁻¹¹ meter² second⁻¹.

19. The method of claim 1, wherein viscosity of the diffusivity contrast agent in blood is about 1×10⁻⁴ pascal·second to 1×10⁻³ pascal·second.

20. The method of claim 1, wherein viscosity of the diffusivity contrast agent in blood is about 1×10⁻³ pascal·second to 1×10⁻² pascal·second.

21. The method of claim 1, wherein the diffusivity contrast agent is administered to the subject rectally, orally, nasally, intravenously, by infusion, or intraperitoneally.

22. The method of claim 1, further comprising at least one of performing a risk assessment of a disease, diagnosing a disease, monitoring disease therapy, staging a disease, and therapy evaluation targeting a biological barrier based on characterization of the transport of the diffusivity contrast agent in the region of interest.

23. The method of claim 1, wherein the diffusivity contrast agent is a therapeutic drug.

24. The method of claim 23, further comprising identifying and/or quantifying delivery of the therapeutic drug.
